(19) United States
(12) Patent Application Publication
(10) Pub. No.: US 2026/0108224 A1
(43) Pub. Date: Apr. 23, 2026

(54) METHOD FOR USING ANATOMICAL LANDMARKS OF BALANCE ORGAN TO CONSTRUCT THREE-DIMENSIONAL CEPHALOMETRIC COORDINATE SYSTEM

(71) Applicant: Institute of Automation, Chinese Academy of Sciences, Beijing (CN)

(72) Inventors: Qi Wang, Beijing (CN); Hui Chang, Beijing (CN); Xiaoguang Zhao, Beijing (CN); Shiying Sun, Beijing (CN); Yujia Zhang, Beijing (CN); Kuo Wan, Beijing (CN)

(21) Appl. No.: 19/168,510

(22) PCT Filed: Feb. 27, 2024
(86) PCT No.: PCT/CN2024/078726

§ 371 Date: Sep. 24, 2025

(30) Foreign Application Priority Data
Jan. 30, 2024 (CN) 202410126189.2

(51) Int. Cl. A61B 6/50; A61B 6/00; A61B 34/10; G06T 15/00

(52) U.S. Cl. CPC A61B 6/501; A61B 6/5211; A61B 34/10; G06T 15/00; A61B 2034/105; A61B 2034/107; G06T 2207/10081; G06T 2207/20081; G06T 2207/20084; G06T 2207/30016

(57) ABSTRACT

A method for using anatomical landmarks of a balance organ to construct a three-dimensional cephalometric coordinate system includes acquiring a CT image of a craniofacial region to be measured. The anatomical landmarks of the CT image are determined. On the basis thereof, a horizontal plane, and then a coronal plane and a median sagittal-plane plane are determined. The horizontal plane is a plane formed by certain of the anatomical landmarks. The coronal plane is determined using the horizontal plane and anatomical landmarks, and the median sagittal plane is determined using an anatomical landmark and both the horizontal and coronal. An intersection point of the three planes is used as the origin of a three-dimensional cephalometric coordinate system.

### CROSS-REFERENCE TO RELATED APPLICATIONS

This application is a national phase entry under 35 U.S. C. § 371 of International Patent Application PCT/CN2024/078726, filed Feb. 27, 2024, designating the United States of America and published as International Patent Publication WO 2025/161076 A1 on Aug. 7, 2025, which claims the benefit under Article 8 of the Patent Cooperation Treaty of Chinese Patent Application Serial No. 202410126189.2, filed Jan. 30, 2024.

### TECHNICAL FIELD

The present disclosure relates to the field of medical technologies, and, in particular, to a method for constructing three-dimensional cephalometric coordinate system using anatomical landmarks of balance organs.

### BACKGROUND

In the related art, craniofacial plastic surgery and orthodontic treatment often rely on cephalometric measurements. However, traditional two-dimensional cephalometric measurements are no longer sufficient to meet clinical needs when facing the problem of facial asymmetry. To solve this problem, a precise three-dimensional coordinate system of craniofacial anatomical landmarks is needed as guidance and assistance for surgery.

Although computed tomography (CT) data in the related art provides us with this coordinate system, there is no direct relationship between the traditional coordinate system and the object being photographed. This means that when the photographing posture of a patient changes, the three-dimensional coordinate of each point in the CT data also changes, resulting in significant three-dimensional coordinate errors.

In the related art, the anatomical landmarks used in three-dimensional cephalometric measurement still follow the traditional two-dimensional cephalometric measurement, selecting some relatively shallow, growing, and blurred boundary landmarks. Due to the asymmetry between the left and right sides of the face, it is currently impossible to form a unique horizontal plane. In addition, these set planes are not directly related to physiological balance, so they still cannot meet clinical needs.

### BRIEF SUMMARY

The present disclosure provides a method for constructing three-dimensional cephalometric coordinate system using anatomical landmarks of balance organs, which may accurately determine the three-dimensional coordinate information of craniofacial anatomical landmarks to provide quantitative evaluation and guidance for craniofacial surgery and corrective treatment.

The present disclosure provides a method for constructing three-dimensional cephalometric coordinate system using anatomical landmarks of balance organs, including:
obtaining a computed tomography (CT) image of a craniofacial region to be measured;
inputting the CT image of the craniofacial region to be measured into a trained craniofacial key anatomical point recognition model, and obtaining an apex of a dens axis (AODA), an anterior nasal spine (ANS), a helicotrema left (HtL), a helicotrema right (HtR), and a sphenoidal rostrum (SR) of a CT image of the craniofacial region to be measured output by the trained craniofacial key anatomical point recognition model;
based on the AODA, the ANS, the HtL, the HtR, and the SR of the CT image of the craniofacial region to be measured, determining a horizontal plane first, and then determining a coronal plane and a mid-sagittal plane, where the horizontal plane is a plane formed by intersection of a parallel line of a line connecting the AODA and the ANS and a line connecting HtL and HtR, the coronal plane is perpendicular to the horizontal plane and passes through the line connecting HtL and HtR, and the mid-sagittal plane is perpendicular to both the horizontal plane and the coronal plane and passes through the SR; and
establishing a three-dimensional cephalometric coordinate system using an intersection point of the coronal plane, the mid-sagittal plane, and the horizontal plane of the CT image of the craniofacial region to be measured as an origin of the three-dimensional cephalometric coordinate system.

In an embodiment, a training procedure of the craniofacial key anatomical point recognition model includes:
designing a model architecture of the craniofacial key anatomical point recognition model using a deep neural network and a three-dimensional convolutional layer based on a characteristic of biological anatomy;
obtaining a first dataset, where the first dataset includes a plurality of human head CT images with three-dimensional spatial resolution;
annotating the AODA, the ANS, the HtL, the HtR, and the SR in the plurality of human head CT images with three-dimensional spatial resolution; and
training the craniofacial key anatomical point recognition model through deep learning based on an annotated first dataset, until the craniofacial key anatomical point recognition model outputs correct AODA, ANS, HtL, HtR, and SR with any input of craniofacial CT images to the craniofacial key anatomical point recognition model.

In an embodiment, after obtaining the first dataset, the method further includes:
based on the plurality of human head CT images with three-dimensional spatial resolution, obtaining corresponding three-dimensional brain models of the plurality of human head CT images with three-dimensional spatial resolution through a three-dimensional reconstruction algorithm.

In an embodiment, annotating the AODA, the ANS, the HtL, the HtR, and the SR in the plurality of human head CT images with three-dimensional spatial resolution includes:
annotating the AODA, the ANS, the HtL, the HtR, and the SR in the corresponding three-dimensional brain models of the plurality of human head CT images with three-dimensional spatial resolution.

The present disclosure further provides an apparatus for constructing three-dimensional cephalometric coordinate system using anatomical landmarks of balance organs, including:
an obtaining module, used for obtaining a computed tomography (CT) image of a craniofacial region to be measured;
an inputting module, used for inputting the CT image of the craniofacial region to be measured into a trained craniofacial key anatomical point recognition model, and obtaining an apex of a dens axis (AODA), an anterior nasal spine (ANS), a helicotrema left (HtL), a helicotrema right (HtR), and a sphenoidal rostrum (SR) of a CT image of the craniofacial region to be measured output by the trained craniofacial key anatomical point recognition model;
a determining module, used for, based on the AODA, the ANS, the HtL, the HtR, and the SR of the CT image of the craniofacial region to be measured, determining a horizontal plane first, and then determining a coronal plane and a mid-sagittal plane, where the horizontal plane is a plane formed by intersection of a parallel line of a line connecting the AODA and the ANS and a line connecting HtL and HtR, the coronal plane is perpendicular to the horizontal plane and passes through the line connecting HtL and HtR, and the mid-sagittal plane is perpendicular to both the horizontal plane and the coronal plane and passes through the SR; and
an establishing module, used for establishing a three-dimensional cephalometric coordinate system using an intersection point of the coronal plane, the mid-sagittal plane, and the horizontal plane of the CT image of the craniofacial region to be measured as an origin of the three-dimensional cephalometric coordinate system.

The present disclosure further provides an electronic device, including a memory, a processor, and a computer program stored on the memory and executable on the processor. When executing the computer program, the processor performs any of the methods for constructing three-dimensional cephalometric coordinate system using anatomical landmarks of balance organs as described above.

The present disclosure further provides a non-transient computer-readable storage medium, on which a computer program is stored. When executed by a processor, the computer program performs any of the method for constructing three-dimensional cephalometric coordinate system using anatomical landmarks of balance organs as described above.

The present disclosure further provides a computer program product, including a computer program that, when executed by a processor, performs any of the method for constructing three-dimensional cephalometric coordinate system using anatomical landmarks of balance organs as described above.

The present disclosure provides a method for constructing three-dimensional cephalometric coordinate system using anatomical landmarks of balance organs. Key anatomical landmarks such as AODA, ANS, HtL, HtR, and SR in craniofacial CT images are selected. Based on these landmarks, the coronal plane, the mid-sagittal plane, and the horizontal plane are determined. Then, the intersection point of the three planes is used as the origin of the three-dimensional cephalometric coordinate system to establish a three-dimensional cephalometric coordinate system, which enables each point in the CT data to obtain a relatively stable coordinate value.

### BRIEF DESCRIPTION OF THE DRAWINGS

To provide a clearer explanation of the solutions in the embodiments of the present disclosure, a brief introduction is given to the accompanying drawings required for the description of the embodiments. The accompanying drawings in the following description are some embodiments of the present disclosure. For those skilled in the art, other drawings may be obtained based on these drawings without creative labor.

FIG. 1 is a schematic flowchart of a method for constructing three-dimensional cephalometric coordinate system using anatomical landmarks of balance organs according to an embodiment of the present disclosure;

FIG. 2 is a schematic structural diagram of an apparatus for constructing three-dimensional cephalometric coordinate system using anatomical landmarks of balance organs according to an embodiment of the present disclosure; and

FIG. 3 is a schematic structural diagram of an electronic device according to an embodiment of the present disclosure.

### DETAILED DESCRIPTION

To make the objective, solution, and effects of the present disclosure clearer, the following provides a clear and complete description of the solution in the present disclosure in conjunction with the accompanying drawings. The described embodiments are part of the embodiments of the present disclosure, not all of them. Based on the embodiments in the present disclosure, all other embodiments obtained by those skilled in the art without creative labor fall within the scope of protection of the present disclosure.

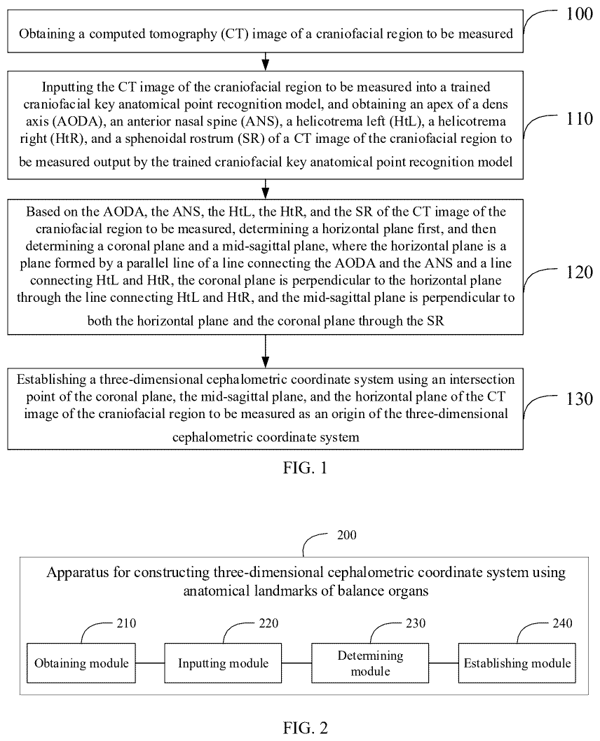

The method for constructing three-dimensional cephalometric coordinate system using anatomical landmarks of balance organs provided in the present disclosure is described below in conjunction with FIG. 1.

FIG. 1 is a schematic flowchart of a method for constructing three-dimensional cephalometric coordinate system using anatomical landmarks of balance organs according to an embodiment of the present disclosure. As shown in FIG. 1, the method includes the following steps.

Step 100: obtaining a computed tomography (CT) image of a craniofacial region to be measured.

In an embodiment, to establish a three-dimensional cephalometric coordinate system for the craniofacial region to be measured, a CT image of the craniofacial region to be measured may be obtained first.

In an embodiment, a CT imaging apparatus may be used to image a human head to obtain a CT image of the craniofacial region to be measured.

In an embodiment, an existing CT image of the craniofacial region to be measured may be obtained from hospitals or research institutions.

In an embodiment, the CT image of the craniofacial region to be measured may have three-dimensional spatial resolution.

Step 110: inputting the CT image of the craniofacial region to be measured into a trained craniofacial key anatomical point recognition model, and obtaining an apex of a dens axis (AODA), an anterior nasal spine (ANS), a helicotrema left (HtL), a helicotrema right (HtR), and a sphenoidal rostrum (SR) of a CT image of the craniofacial region to be measured output by the trained craniofacial key anatomical point recognition model.

In an embodiment, to establish a relatively stable three-dimensional coordinate system for craniofacial imaging, some anatomical landmarks that are relatively stable, clear, and naturally single points during development may be selected from the craniofacial CT image, namely the AODA, the ANS, the HtL, the HtR, and the SR. Based on these anatomical landmarks, a three-dimensional cephalometric coordinate system may be established, which not only conforms to the natural head position of human physiology, but also avoids the defect of lacking left-right symmetry in a two-dimensional measurement.

In an embodiment, the anatomical points selected in the present disclosure conforms to physiology and a head position established based on balance organs is horizontal. The present disclosure uses the balance organ as a reference plane and selects a helicotrema to represent an inner ear, which is an important component of balance organs; the line connecting the ANS and the AODA represents this horizontal plane, and a skull midline passes through the relatively most stable SR in the skull.

In an embodiment, to accurately and automatically determine the AODA, the ANS, the HtL, the HtR, and the SR in CT image of the craniofacial region to be measured, a craniofacial key anatomical point recognition model may be trained through deep learning, and the trained craniofacial key anatomical point recognition model may automatically determine the AODA, the ANS, the HtL, the HtR, and the SR of the input CT image of the craniofacial region to be measured.

In an embodiment, the CT image of the craniofacial region to be measured may be input into a trained craniofacial key anatomical point recognition model to obtain a CT image annotated with AODA, ANS, HtL, HtR, and SR points output by the trained craniofacial key anatomical point recognition model, which facilitates the subsequent construction of a coronal plane, a mid-sagittal plane, and a horizontal plane.

In an embodiment, AODA is a bony protrusion located above and behind the axis, and aligns with a depression in the posteromedian of a first cervical vertebra.

In an embodiment, ANS is a sharp bony protrusion located between a lower edge of a nasal piriform aperture and a maxilla.

In an embodiment, HtL and HtR are small holes connecting a tympanic scale and a vestibular scale, and the HtL and HtR are located at a central top of a cochlea.

In an embodiment, SR is a triangular spine on a lower surface of a sphenoid bone, and the SR is connected to a sphenoid crest at an anterior surface of the sphenoid bone and received by the vomerine groove of a vomer.

Step 120, based on the AODA, the ANS, the HtL, the HtR, and the SR of the CT image of the craniofacial region to be measured, determining a horizontal plane first, and then determining a coronal plane and a mid-sagittal plane, where the horizontal plane is a plane formed by intersection of a parallel line of a line connecting the AODA and the ANS and a line connecting HtL and HtR, the coronal plane is perpendicular to the horizontal plane and passes through the line connecting HtL and HtR, and the mid-sagittal plane is perpendicular to both the horizontal plane and the coronal plane and passes through the SR.

In an embodiment, after determining the points of AODA, ANS, HtL, HtR, and SR in the CT image of the craniofacial region to be measured, the coronal plane, the mid-sagittal plane, and the horizontal plane may be constructed based on these points.

In an embodiment, the horizontal plane is a plane formed by intersection of a parallel line of a line connecting the AODA and the ANS and a line connecting HtL and HtR.

In an embodiment, the coronal plane is perpendicular to the horizontal plane and passes through the line connecting HtL and HtR.

In an embodiment, the mid-sagittal plane is perpendicular to both the horizontal plane and the coronal plane and passes through the SR.

Step 130: establishing a three-dimensional cephalometric coordinate system using an intersection point of the coronal plane, the mid-sagittal plane, and the horizontal plane of the CT image of the craniofacial region to be measured as an origin of the three-dimensional cephalometric coordinate system.

In an embodiment, after determining the horizontal plane, the coronal plane, and the mid-sagittal plane of the CT image of the craniofacial region to be measured, an intersection point is generated of the three planes. This intersection point may be used as the origin of the three-dimensional cephalometric coordinate system to establish a three-dimensional cephalometric coordinate system. Based on this three-dimensional coordinate system, each point in the existing craniofacial CT data may obtain a relatively stable coordinate value, making the measurement data required for diagnosis and planning of orthodontic, orthognathic, and implant in each case more convenient, intuitive, and accurate, avoiding errors caused by projection angles and measurement habits in traditional two-dimensional measurements.

The present disclosure provides a method for constructing three-dimensional cephalometric coordinate system using anatomical landmarks of balance organs. Key anatomical landmarks such as AODA, ANS, HtL, HtR, and SR in craniofacial CT images are selected. Based on these landmarks, the coronal plane, the mid-sagittal plane, and the horizontal plane are determined. Then, the intersection point of the three planes is used as the origin of the three-dimensional cephalometric coordinate system to establish a three-dimensional cephalometric coordinate system, which enables each point in the CT data to obtain a relatively stable coordinate value.

In an embodiment, a training procedure of the craniofacial key anatomical point recognition model includes:
designing a model architecture of the craniofacial key anatomical point recognition model using a deep neural network and a three-dimensional convolutional layer based on a characteristic of biological anatomy;
obtaining a first dataset, where the first dataset includes a plurality of human head CT images with three-dimensional spatial resolution;
annotating the AODA, the ANS, the HtL, the HtR, and the SR in the plurality of human head CT images with three-dimensional spatial resolution; and
training the craniofacial key anatomical point recognition model through deep learning based on an annotated first dataset, until the craniofacial key anatomical point recognition model outputs correct AODA, ANS, HtL, HtR, and SR with any input of craniofacial CT images to the craniofacial key anatomical point recognition model.

In an embodiment, to train a craniofacial key anatomical point recognition model, a model architecture of the craniofacial key anatomical point recognition model is designed using a deep neural network and a three-dimensional convolutional layer based on a characteristic of biological anatomy.

In an embodiment, a dataset may be obtained, which may include a plurality of human head CT images with three-dimensional spatial resolution.

In an embodiment, a plurality of existing human head CT images with three-dimensional spatial resolution may be obtained from hospitals or research institutions, or a plurality of human head CT images with three-dimensional spatial resolution may be artificially created, or the first dataset may be obtained through other means, which is not limited in the present disclosure.

In an embodiment, after obtaining the first dataset, the AODA, the ANS, the HtL, the HtR, and the SR may be annotated in each human head CT image to enable the craniofacial key anatomical point recognition model to identify these key anatomical points.

In an embodiment, multimodal fusion of CT image, red green blue-depth (RGB-D) sensor information, and expert annotation information may be used to train the craniofacial key anatomical point recognition model.

In an embodiment, the craniofacial key anatomical point recognition model may be trained based on the annotated first dataset through deep learning until the craniofacial key anatomical point recognition model outputs correct AODA, ANS, HtL, HR, and SR with any input of craniofacial CT images to the craniofacial key anatomical point recognition model.

In an embodiment, prior knowledge of craniofacial shape and structure may be combined to introduce set constraints on bone structure, optimize a loss function, and ensure that the craniofacial key anatomical points generated by the model conform to actual anatomy.

In an embodiment, the algorithm used in deep learning may be a back propagation algorithm, a convolutional neural network, a recurrent neural network, or other algorithms that may achieve training effects, which is not limited in the present disclosure.

In an embodiment, after the training of the craniofacial key anatomical point recognition model is completed, the CT image of the craniofacial region to be measured may be input into a trained craniofacial key anatomical point recognition model, which may automatically and accurately determine the AODA, the ANS, the HtL, the HtR, and the SR in the CT image.

The present disclosure provides a method for constructing three-dimensional cephalometric coordinate system using anatomical landmarks of balance organs. The CT image of the craniofacial region to be measured is input into a trained craniofacial key anatomical point recognition model to automatically and accurately determine the AODA, the ANS, the HtL, the HtR, and the SR in the CT image, which facilitates the subsequent establishment of a three-dimensional cephalometric coordinate system, and improves the efficiency of establishment.

In an embodiment, after obtaining the first dataset, the method further includes:
based on the plurality of human head CT images with three-dimensional spatial resolution, obtaining corresponding three-dimensional brain models of the plurality of human head CT images with three-dimensional spatial resolution through a three-dimensional reconstruction algorithm.

In an embodiment, since the human head CT image is a two-dimensional image, to annotate the specific three-dimensional position of the AODA, the ANS, the HtL, the HtR, and the SR in the CT image, corresponding three-dimensional brain models of the plurality of human head CT images with three-dimensional spatial resolution may be obtained through a three-dimensional reconstruction algorithm.

In an embodiment, the three-dimensional reconstruction algorithm may be a method of triangulation, monocular vision, structured light, or other three-dimensional reconstruction algorithms, which is not limited in the present disclosure.

The present disclosure provides a method for constructing three-dimensional cephalometric coordinate system using anatomical landmarks of balance organs. A corresponding three-dimensional brain model is obtained based on human head CT images through a three-dimensional reconstruction algorithm, which facilitates the subsequent annotation of the AODA, the ANS, the HtL, the HtR, and the SR in the three-dimensional model.

In an embodiment, annotating the AODA, the ANS, the HtL, the HtR, and the SR in the plurality of human head CT images with three-dimensional spatial resolution includes:
annotating the AODA, the ANS, the HtL, the HtR, and the SR in the corresponding three-dimensional brain models of the plurality of human head CT images with three-dimensional spatial resolution.

In an embodiment, after obtaining the corresponding three-dimensional brain models of the plurality of human head CT images with three-dimensional spatial resolution, the AODA, the ANS, the HtL, the HtR, and the SR may be accurately annotated in the three-dimensional brain model, which facilitates subsequent training of the craniofacial key anatomical point recognition model.

In an embodiment, the present disclosure has manually tested a large number of actual cases in clinical practice. The coordinate system may be used for diagnosis and treatment design, which facilitates discovery of difficult clinical causes. The procedure may be qualitative, quantitative, and in line with scientific principles, and may also allow patients to intuitively understand their own conditions and causes, which facilitates communication with patients. The use of the coordinate system helps to form clear treatment designs, especially for guiding invisible orthodontic teeth alignment, orthognathic surgery, plastic surgery, orthopedics, temporomandibular joint occlusion reconstruction treatment, etc.

The present disclosure provides a method for constructing three-dimensional cephalometric coordinate system using anatomical landmarks of balance organs. The AODA, the ANS, the HtL, the HtR, and the SR are annotated in the corresponding three-dimensional brain models of the plurality of human head CT images with three-dimensional spatial resolution, which facilitates subsequent training of the craniofacial key anatomical point recognition model.

The following describes an apparatus for constructing three-dimensional cephalometric coordinate system using anatomical landmarks of balance organs provided in the present disclosure.

FIG. 2 is a schematic structural diagram of an apparatus 200 for constructing three-dimensional cephalometric coordinate system using anatomical landmarks of balance organs according to an embodiment of the present disclosure. As shown in FIG. 2, the apparatus 200 includes an obtaining module 210, an inputting module 220, a determining module 230, and an establishing module 240.

The obtaining module 210 is used for obtaining a computed tomography (CT) image of a craniofacial region to be measured.

The inputting module 220 is used for inputting the CT image of the craniofacial region to be measured into a trained craniofacial key anatomical point recognition model, and obtaining an apex of a dens axis (AODA), an anterior nasal spine (ANS), a helicotrema left (HtL), a helicotrema right (HtR), and a sphenoidal rostrum (SR) of a CT image of the craniofacial region to be measured output by the trained craniofacial key anatomical point recognition model.

The determining module 230 is used for, based on the AODA, the ANS, the HtL, the HtR, and the SR of the CT image of the craniofacial region to be measured, determining a horizontal plane first, and then determining a coronal plane and a mid-sagittal plane, where the horizontal plane is a plane formed by intersection of a parallel line of a line connecting the AODA and the ANS and a line connecting HtL and HtR, the coronal plane is perpendicular to the horizontal plane and passes through the line connecting HtL and HtR, and the mid-sagittal plane is perpendicular to both the horizontal plane and the coronal plane and passes through the SR.

The establishing module 240 is used for establishing a three-dimensional cephalometric coordinate system using an intersection point of the coronal plane, the mid-sagittal plane, and the horizontal plane of the CT image of the craniofacial region to be measured as an origin of the three-dimensional cephalometric coordinate system.

The present disclosure provides an apparatus for constructing three-dimensional cephalometric coordinate system using anatomical landmarks of balance organs. Key anatomical landmarks such as AODA, ANS, HtL, HtR, and SR in craniofacial CT images are selected. Based on these landmarks, the coronal plane, the mid-sagittal plane, and the horizontal plane are determined. Then, the intersection point of the three planes is used as the origin of the three-dimensional cephalometric coordinate system to establish a three-dimensional cephalometric coordinate system, which enables each point in the CT data to obtain a relatively stable coordinate value.

The apparatus for constructing three-dimensional cephalometric measurement coordinate system using anatomical landmarks of balance organs provided in the present disclosure corresponds to the method for constructing three-dimensional cephalometric measurement coordinate system using anatomical landmarks of balance organs provided in the above embodiments. The relevant features of the apparatus for constructing three-dimensional cephalometric measurement coordinate system using anatomical landmarks of balance organs provided in the present disclosure may refer to the relevant features of the method for constructing three-dimensional cephalometric measurement coordinate system using anatomical landmarks of balance organs provided in the above embodiments, and are not repeated here.

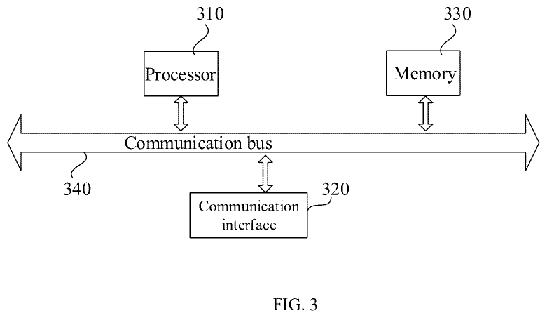

FIG. 3 is a schematic structural diagram of an electronic device according to an embodiment of the present disclosure. As show in FIG. 3, the electronic device may include: a processor 310, a communication interface 320, a memory 330, and a communication bus 340, where the processor 310, the communication interface 320, and the memory 330 communicate with each other through the communication bus 340. The processor 310 may call logical instructions in the memory 330 to perform the method for constructing three-dimensional cephalometric coordinate system using anatomical landmarks of balance organs. The method includes: obtaining a computed tomography (CT) image of a craniofacial region to be measured; inputting the CT image of the craniofacial region to be measured into a trained craniofacial key anatomical point recognition model, and obtaining an apex of a dens axis (AODA), an anterior nasal spine (ANS), a helicotrema left (HtL), a helicotrema right (HtR), and a sphenoidal rostrum (SR) of a CT image of the craniofacial region to be measured output by the trained craniofacial key anatomical point recognition model; based on the AODA, the ANS, the HtL, the HtR, and the SR of the CT image of the craniofacial region to be measured, determining a horizontal plane first, and then determining a coronal plane and a mid-sagittal plane, where the horizontal plane is a plane formed by intersection of a parallel line of a line connecting the AODA and the ANS and a line connecting HtL and HtR, the coronal plane is perpendicular to the horizontal plane and passes through the line connecting HtL and HtR, and the mid-sagittal plane is perpendicular to both the horizontal plane and the coronal plane and passes through the SR; and establishing a three-dimensional cephalometric coordinate system using an intersection point of the coronal plane, the mid-sagittal plane, and the horizontal plane of the CT image of the craniofacial region to be measured as an origin of the three-dimensional cephalometric coordinate system.

In addition, the logical instructions in the above-mentioned memory 330 may be implemented in the form of software functional unit and stored in a computer-readable storage medium when sold or used as an independent product. Based on such understanding, the solutions of the present disclosure in essence or a part of the technical solutions that contributes to the prior art, or all or part of the solutions, may be embodied in the form of a software product, which is stored in a storage medium, including several instructions to cause a computer device (which may be a personal computer, server, or network device, etc.) or a processor to perform all or part of the steps of the methods described in the respective embodiments of the present disclosure. The storage medium described above includes various media that may store a program code such as a U disk, a mobile hard disk, a read-only memory (ROM), a random access memory (RAM), a magnetic disk, or a compact disk.

The present disclosure further provides a computer program product including a computer program that may be stored on a non-transient computer-readable storage medium. When the computer program is executed by a processor, the computer may perform the methods provided by the above methods for constructing three-dimensional cephalometric coordinate system using anatomical landmarks of balance organs. The method includes: obtaining a computed tomography (CT) image of a craniofacial region to be measured; inputting the CT image of the craniofacial region to be measured into a trained craniofacial key anatomical point recognition model, and obtaining an apex of a dens axis (AODA), an anterior nasal spine (ANS), a helicotrema left (HtL), a helicotrema right (HtR), and a sphenoidal rostrum (SR) of a CT image of the craniofacial region to be measured output by the trained craniofacial key anatomical point recognition model; based on the AODA, the ANS, the HtL, the HtR, and the SR of the CT image of the craniofacial region to be measured, determining a horizontal plane first, and then determining a coronal plane and a mid-sagittal plane, where the horizontal plane is a plane formed by intersection of a parallel line of a line connecting the AODA and the ANS and a line connecting HtL and HtR, the coronal plane is perpendicular to the horizontal plane and passes through the line connecting HtL and HtR, and the mid-sagittal plane is perpendicular to both the horizontal plane and the coronal plane and passes through the SR; and establishing a three-dimensional cephalometric coordinate system using an intersection point of the coronal plane, the mid-sagittal plane, and the horizontal plane of the CT image of the craniofacial region to be measured as an origin of the three-dimensional cephalometric coordinate system.

The present disclosure further provides a non-transient computer-readable storage medium on which a computer program is stored. When executed by a processor, the computer program implements the method for constructing three-dimensional cephalometric coordinate system using anatomical landmarks of balance organs provided by the above methods. The method includes: obtaining a computed tomography (CT) image of a craniofacial region to be measured; inputting the CT image of the craniofacial region to be measured into a trained craniofacial key anatomical point recognition model, and obtaining an apex of a dens axis (AODA), an anterior nasal spine (ANS), a helicotrema left (HtL), a helicotrema right (HtR), and a sphenoidal rostrum (SR) of a CT image of the craniofacial region to be measured output by the trained craniofacial key anatomical point recognition model; based on the AODA, the ANS, the HtL, the HtR, and the SR of the CT image of the craniofacial region to be measured, determining a horizontal plane first, and then determining a coronal plane and a mid-sagittal plane, where the horizontal plane is a plane formed by intersection of a parallel line of a line connecting the AODA and the ANS and a line connecting HtL and HtR, the coronal plane is perpendicular to the horizontal plane and passes through the line connecting HtL and HtR, and the mid-sagittal plane is perpendicular to both the horizontal plane and the coronal plane and passes through the SR; and establishing a three-dimensional cephalometric coordinate system using an intersection point of the coronal plane, the mid-sagittal plane, and the horizontal plane of the CT image of the craniofacial region to be measured as an origin of the three-dimensional cephalometric coordinate system.

The apparatus embodiments described above are only schematic, where the units described as separate components may be or may not be physically separated, and the components displayed as units may be or may not be physical units, that is, they may be located in one place or distributed across a plurality of network units. Some or all modules may be selected according to actual needs to achieve the purpose of this embodiment. Those skilled in the art may understand and implement without creative labor.

Through the description of the above implementation, those skilled in the art may clearly understand that each implementation may be achieved through software and necessary universal hardware platforms, and it may also be achieved through hardware. Based on such understanding, the solutions mentioned above, or the parts that contribute to the related art, may be reflected in the form of software product, which may be stored in computer-readable storage media such as ROM/RAM, magnetic disks, optical disks, etc., including several instructions to enable a computer device (which may be a personal computer, a server, or a network device, etc.) to perform the methods described in various embodiments or certain parts of the embodiments.

Finally, it should be noted that the above embodiments are only used to illustrate the solutions of the present disclosure, and not to limit them; although the present disclosure has been described in detail with reference to the aforementioned embodiments, those skilled in the art should understand that they may still modify the solutions described in the aforementioned embodiments, or replace some of the features equally; these modifications or substitutions do not deviate from the essence of the corresponding solutions from the scope of the technical solutions of the various embodiments of the present disclosure.

1-8. (canceled)

9. A method for constructing three-dimensional cephalometric coordinate system using anatomical landmarks of balance organs, comprising:
obtaining a computed tomography (CT) image of a craniofacial region to be measured;
inputting the CT image of the craniofacial region to be measured into a trained craniofacial key anatomical point recognition model, and obtaining an apex of a dens axis (AODA), an anterior nasal spine (ANS), a helicotrema left (HtL), a helicotrema right (HtR), and a sphenoidal rostrum (SR) of a CT image of the craniofacial region to be measured output by the trained craniofacial key anatomical point recognition model;
based on the AODA, the ANS, the HtL, the HtR, and the SR of the CT image of the craniofacial region to be measured, determining a horizontal plane first, and then determining a coronal plane and a mid-sagittal plane, wherein the horizontal plane is a plane formed by intersection of a parallel line of a line connecting the AODA and the ANS and a line connecting HtL and HtR, the coronal plane is perpendicular to the horizontal plane and passes through the line connecting HtL and HtR, and the mid-sagittal plane is perpendicular to both the horizontal plane and the coronal plane and passes through the SR; and
establishing a three-dimensional cephalometric coordinate system using an intersection point of the coronal plane, the mid-sagittal plane, and the horizontal plane of the CT image of the craniofacial region to be measured as an origin of the three-dimensional cephalometric coordinate system;
wherein a training procedure of the craniofacial key anatomical point recognition model comprises:
designing a model architecture of the craniofacial key anatomical point recognition model using a deep neural network and a three-dimensional convolutional layer based on a characteristic of biological anatomy;
obtaining a first dataset, wherein the first dataset comprises a plurality of human head CT images with three-dimensional spatial resolution;
annotating the AODA, the ANS, the HtL, the HtR, and the SR in the plurality of human head CT images with three-dimensional spatial resolution; and
training the craniofacial key anatomical point recognition model through deep learning based on an annotated first dataset, until the craniofacial key anatomical point recognition model outputs correct AODA, ANS, HtL, HtR, and SR with any input of craniofacial CT images to the craniofacial key anatomical point recognition model.

10. The method of claim 9, wherein after obtaining the first dataset, the method further comprises:
based on the plurality of human head CT images with three-dimensional spatial resolution, obtaining corresponding three-dimensional brain models of the plurality of human head CT images with three-dimensional spatial resolution through a three-dimensional reconstruction algorithm.

11. The method of claim 10, wherein annotating the AODA, the ANS, the HtL, the HtR, and the SR in the plurality of human head CT images with three-dimensional spatial resolution comprises:
annotating the AODA, the ANS, the HtL, the HtR, and the SR in the corresponding three-dimensional brain models of the plurality of human head CT images with three-dimensional spatial resolution.

12. An apparatus for constructing three-dimensional cephalometric coordinate system using anatomical landmarks of balance organs, comprising:
an obtaining module, used for obtaining a computed tomography (CT) image of a craniofacial region to be measured;
an inputting module, used for inputting the CT image of the craniofacial region to be measured into a trained craniofacial key anatomical point recognition model, and obtaining an apex of a dens axis (AODA), an anterior nasal spine (ANS), a helicotrema left (HtL), a helicotrema right (HtR), and a sphenoidal rostrum (SR) of a CT image of the craniofacial region to be measured output by the trained craniofacial key anatomical point recognition model;
a determining module, used for, based on the AODA, the ANS, the HtL, the HtR, and the SR of the CT image of the craniofacial region to be measured, determining a horizontal plane first, and then determining a coronal plane and a mid-sagittal plane, wherein the horizontal plane is a plane formed by intersection of a parallel line of a line connecting the AODA and the ANS and a line connecting HtL and HtR, the coronal plane is perpendicular to the horizontal plane and passes through the line connecting HtL and HtR, and the mid-sagittal plane is perpendicular to both the horizontal plane and the coronal plane and passes through the SR; and
an establishing module, used for establishing a three-dimensional cephalometric coordinate system using an intersection point of the coronal plane, the mid-sagittal plane, and the horizontal plane of the CT image of the craniofacial region to be measured as an origin of the three-dimensional cephalometric coordinate system;
wherein a training procedure of the craniofacial key anatomical point recognition model comprises:
designing a model architecture of the craniofacial key anatomical point recognition model using a deep neural network and a three-dimensional convolutional layer based on a characteristic of biological anatomy;
obtaining a first dataset, wherein the first dataset comprises a plurality of human head CT images with three-dimensional spatial resolution;
annotating the AODA, the ANS, the HtL, the HtR, and the SR in the plurality of human head CT images with three-dimensional spatial resolution; and
training the craniofacial key anatomical point recognition model through deep learning based on an annotated first dataset, until the craniofacial key anatomical point recognition model outputs correct AODA, ANS, HtL, HtR, and SR with any input of craniofacial CT images to the craniofacial key anatomical point recognition model.

13. An electronic device, comprising a memory, a processor, and a computer program stored on the memory and executable on the processor, wherein the processor, when executing the computer program, performs the method for constructing three-dimensional cephalometric coordinate system using anatomical landmarks of balance organs of claim 9.

14. A non-transient computer-readable storage medium, storing a computer program, wherein the computer program, when executed by a processor, performs the method for constructing three-dimensional cephalometric coordinate system using anatomical landmarks of balance organs of claim 9.

15. A computer program product, comprising a computer program, wherein the computer program, when executed by a processor, performs the method for constructing three-dimensional cephalometric coordinate system using anatomical landmarks of balance organs of claim 9.
